(19) United States
(12) Patent Application Publication
(10) Pub. No.: US 2003/0021783 A1
(43) Pub. Date: Jan. 30, 2003

(54) Sepsis remedy comprising anti-IL-8 antibody as active ingredient

(76) Inventors: Masaki Kitajima, Tokyo (JP); Go Wakabayashi, Tokyo (JP); Kouji Matsushima, Chiba (JP)

Correspondence Address: Bruce D. Grant, Morrison & Foerster LLP, Suite 500, 3811 Valley Centre Drive, San Diego, CA, 92130, US

(21) Appl. No.: 10/247,462

(22) Filed: Sep. 18, 2002

Related U.S. Application Data
(63) Continuation of application No. 09/284,781, filed on Jun. 1, 1999, now abandoned.
Application No. 09/284,781 is a 371 of international application No. PCT/JP97/01963, filed on Jun. 9, 1997.

(30) Foreign Application Priority Data
Oct. 22, 1996 (JP) 8-315377

(51) Int. Cl. A61K 39/395

(52) U.S. Cl. 424/145.1; 530/388.23

(57) ABSTRACT

The present invention discloses a therapeutic agent for sepsis, and particularly septic shock, an agent for improving decreased arterial pressure of septic shock, and an agent for relieving increased respiration rate of septic shock, all containing for their active ingredient anti-IL-8 antibody.

### TECHNICAL FIELD

The present invention relates to a therapeutic agent for sepsis and septic shock containing as its active ingredient an anti-Interleukin-8 (IL-8) antibody.

### BACKGROUND ART

IL-8 is a protein belonging to C-X-C chemokine sub-family. It was formerly named monocyte-derived neutrophil chemotactic factor, neutrophil attractant/activation protein-1, neutrophil activating factor and so forth. IL-8 is a factor that induces neutrophil activation and migration, and is produced by various cells due to stimulation of IL-1β, TNF-α and other inflammatory cytokines (Koch, A. E. et al., J. Investig. Med. (1995) 43, 28-38; Larsen, C. G. et al., Immunology (1989) 68, 31-36), PMA, LPS and other mitogens (Yoshimura, T. et al., Proc. Natl. Acad. Sci. U.S.A. (1987) 84, 9233-9237), and cadmium and other heavy metals (Horiguchi, H. et al., Lymphokine Cytokine Res. (1993) 12, 421-428). In addition, hypoxic human umbilical vein endothelial cells are also known to express IL-8 (Karakurum, M. et al., J. Clin. Invest. (1994) 93, 1564-1570).

In order for IL-8 to express its biological activity, it is necessary for IL-8 to bind to IL-8 receptor and stimulate cells expressing IL-8 receptors. IL-8 receptors, which transmit signals inside cells following binding of IL-8, have already been cloned, and their amino acid sequences have been determined. Human IL-8 receptors include receptor referred to as IL-8 receptor A (α or 2) and receptor referred to as IL-8 receptor B (β or 1) (Murphy, P. M. and Tiffany, H. L., Science (1991) 253, 1280-1283; Holmes, W. E. et al., Science (1991) 253, 1278-1280). Both are assumed to have a structure that penetrates the cell membrane 7 times, both are associated with GTP-binding protein in the cytoplasmic domain (Horuk, R., Trends Pharmacol. Sci. (1994) 15, 159-165), and transmit IL-8 signals within cells. Thus, it is possible to inhibit the biological activity of IL-8 by inhibiting binding between IL-8 and IL-8 receptors.

A joint consensus conference was held in 1991 by the Society of Critical Care Medicine and the American College of Chest Physicians. The disease concept of systemic inflammatory response syndrome (SIRS) was advocated at this conference. Namely, a pathological state having any two or more clinical symptoms of the four diagnostic parameters indicated below is diagnosed as the response of the body to trauma, burns, severe pancreatitis, infection or other forms of invasion (Bone, R. C. et al., Chest (1992) 101, 1644-1655).

(1) High body temperature of at least 38° C. or low body temperature below 36° C.

(2) Heart rate of at least 90 beats/minute

(3) Respiration rate of at least 20 breaths/minute or PaCO2 (arterial blood carbon dioxide partial pressure) of less than 32 torr

(4) WBC count of at least 12,000/μl or less than 4,000/μl, or immature WBC count of at least 10%

Sepsis is a disease that presents with any two or more clinical findings of the four diagnostic parameters of SIRS described above that is caused by infection. The pathogen that causes the infection may or may not be confirmed. Trauma, burns and severe pancreatitis are distinguished from sepsis in that the direct cause is not infection.

In addition, septic shock is a disease accompanied by perfusion abnormalities such as low blood pressure even though an adequate amount of circulating body fluids is maintained. As sepsis progresses, there is onset of septic shock within several hours, presenting with decreased systemic peripheral vascular resistance, decreased myocardial contractile force, peripheral circulatory insufficiency, decreased blood pressure and so forth.

The production of cytokines including inflammatory cytokines such as IL-1β, IL-6, IL-8 and TNF-α (Thijs, L. G. and Hack, C. E., Intensive Care Med. (1995) 21 Suppl. 2, 258-263) and chemokines such as IL-8, MCP-1, MCP-2 and MIP-1α has been reported to be increased in the serum or plasma of sepsis patients (Bossink, A. W. et al., Blood (1995) 86, 3841-3847; Fukushima, S. et al., Intensive Care Med. (1996) 22, 1169-1175). In addition, besides these cytokines, eicosanoids such as leukotriene B4, thromboxane B2 and prostaglandins have been reported to be higher than normal, while the complement system has also been reported to be activated (Takakuwa, T. et al., Res. Commun. Chem. Pathol. Pharmacol. (1994) 84, 291-300).

As has been described above, there are multiple types of factors involved as attacking factors of sepsis, and the disease state of sepsis is assumed to be determined through a complex relationship of these factors. Thus, it has previously been completely unknown that anti-IL-8 antibody has therapeutic effects against sepsis and septic shock.

### DISCLOSURE OF THE INVENTION

At present, detection of the site of infection in the body, surgical drainage or excision, and antibiotic therapy are employed for sepsis. In addition, vasopressor agents and steroids are used against septic shock (Figured Pathological Internal Medicine, Vol. 17, Infections, Medical Review, 96-97). However, the overall mortality rate of sepsis patients is still rising to 25-90% even at present (Merk Manual, Japanese language version, 1st edition, Medical Review, 73). This indicates that there are limitations on the efficacy of these therapeutic methods and agents. Thus, there is a need to develop an effective therapeutic agent.

The object of the present invention is to provide a new therapeutic agent for this disease.

As a result of earnest repeated research to provide such a therapeutic agent, the inventors of the present invention found that this object is achieved by anti-IL-8 antibody, thereby leading to completion of the present invention.

Namely, the present invention provides a therapeutic agent for sepsis comprising as its active ingredient an anti-IL-8 antibody. The present invention also provides a septic shock therapeutic agent comprising as its active ingredient an anti-IL-8 antibody.

In addition, the present invention provides a therapeutic agent for sepsis or septic shock comprising as its active ingredient an anti-IL-8 monoclonal antibody.

In addition, the present invention provides a therapeutic agent for sepsis or septic shock comprising as its active ingredient an antibody to mammalian IL-8.

In addition, the present invention provides a therapeutic agent for sepsis or septic shock comprising as its active ingredient an antibody to human IL-8.

In addition, the present invention provides a therapeutic agent for sepsis or septic shock comprising as its active ingredient WS-4 antibody.

In addition, the present invention provides a therapeutic agent for sepsis or septic shock comprising as it active ingredient an anti-IL-8 antibody comprising a human antibody constant region.

In addition, the present invention provides a therapeutic agent for sepsis or septic shock comprising as its active ingredient a humanized or chimeric anti-IL-8 antibody.

In addition, the present invention provides a therapeutic agent for sepsis or septic shock comprising as its active ingredient a humanized WS-4 antibody.

In addition, the present invention provides an agent comprising as its active ingredient an anti-IL-8 antibody that improves decreased arterial blood pressure during septic shock.

Moreover, the present invention provides an agent comprising as its active ingredient an anti-IL-8 antibody that relieves an increased respiration rate during septic shock.

### BRIEF DESCRIPTION OF THE DRAWINGS

FIG. 1 is a graph showing the time-based changes in arterial blood pressure from 0 to 240 minutes following administration of an antibody or physiological saline at 0 minutes and administration of LPS or physiological saline from 5 to 25 minutes. During the period indicated by line a, in the anti-IL-8 antibody dose group, control antibody dose group and LPS group, arterial blood pressure decreased significantly (p<0.05) in comparison with the normal group. During the period indicated by line b, the anti-IL-8 antibody dose group demonstrated significant alleviation (p<0.05) of the decrease in arterial blood pressure in comparison with the LPS group. During the period indicated by line c, the anti-IL-8 antibody dose group demonstrated significant alleviation (p<0.05) of the decrease in arterial blood pressure in comparison with the control antibody dose group.

FIG. 2 is a graph showing the time-based changes in respiration rate from 0 to 240 minutes following administration of an antibody or physiological saline at 0 minutes and administration of LPS or physiological saline from 5 to 25 minutes. During the period indicated by line d, a respiration rate increased significantly (p<0.05) in comparison with the normal group in the anti-IL-8 dose group, antibody control dose group and LPS group, respectively (excluding the control antibody dose group at 165 minutes). During the period indicated by line e, the anti-IL-8 antibody dose group demonstrated significant alleviation (p<0.05) of increased respiratory rate in comparison with the LPS group.

FIG. 3 is a graph showing the time-based changes in rectal temperature from 0 to 240 minutes following administration of antibody or physiological saline at 0 minutes and administration of LPS or physiological saline from 5 to 25 minutes.

FIG. 4 is a graph showing the time-based changes in survival rate after 7 days.

### MODE FOR CARRYING OUT THE INVENTION

1. Anti-IL-8 Antibody

There are no limitations on the origin, type (monoclonal or polyclonal) or form of the anti-IL-8 antibody used in the present invention provided it has therapeutic effects against sepsis and septic shock.

The anti-IL-8 antibody used in the present invention can be obtained in the form of polyclonal or monoclonal antibody using known means. Monoclonal antibody derived from mammals is particularly preferable as an anti-IL-8 antibody used in the present invention. Examples of monoclonal antibodies of mammalian origin include antibody produced in hybridoma and recombinant antibody produced in a host transformed with an expression vector containing antibody gene. The anti-IL-8 antibody used in the present invention is an antibody that inhibits the biological activity of IL-8 by binding to IL-8 to inhibit binding of IL-8 to IL-8 receptors expressed in neutrophils and so forth, thereby blocking the signal transmission of IL-8.

Examples of such antibodies include WS-4 antibody (Ko, Y. et al., J. Immunol. Methods (1992) 149, 226-235) and DM/C7 antibody (Mulligan, M. S. et al., J. Immunol. (1993) 150, 5585-5595), or 6G4.2.5 antibody and A5.12.14 antibody (International Patent Application Laid-Open No. WO 95/23865; Boylan, A. M. et al., J. Clin. Invest. (1992) 89, 1257-1267). Particularly preferable examples of these antibodies include WS-4 antibody.

Furthermore, a WS-4 antibody-producing hybridoma cell line was internationally deposited based on the Budapest Treaty as FERM BP-5507 on Apr. 17, 1996 at the National Institute of Bioscience and Human-Technology the Agency of Industrial Science and Technology (1-1, Higashi 1-chome, Tsukuba-shi, Ibaraki) under the name Mouse hybridoma WS-4.

2. Antibody Produced by Hybridoma

Monoclonal antibody can be obtained by preparing a hybridoma in the manner described below by basically using known technology. Namely, hybridoma can be prepared by using IL-8 as sensitizing antigen, immunizing with this sensitizing antigen in accordance with routine immunization methods, fusing the resulting immunocytes with known parent cells according to known cell fusion methods and screening for monoclonal antibody-producing cells according to routine screening methods.

More specifically, monoclonal antibodies should be prepared in the manner described below.

For example, IL-8 used as a sensitizing antigen for antibody acquisition is obtained by using the IL-8 gene/amino acid sequence respectively disclosed in Matsushima, K. et al., J. Exp. Med. (1988) 167, 1883-1893 for human IL-8, in Harada, A. et al., Int. Immunol. (1993) 5, 681-690 for rabbit IL-8, Ishikawa, J. et al., Gene (1993) 131, 305-306 for dog IL-8, in Seow, H. F. et al., Immuno. Cell Biol. (1994) 72, 398-405 for sheep IL-8, in Villinger, F. et al., J. Immunol. (1995) 155, 3946-3954 for monkey IL-8, in Yoshimura, T. and Johnson, D. G., J. Immunol. (1993) 151, 6225-6236 for guinea pig IL-8, and in Goodman, R. B. et al., Biochemistry (1992) 31, 10483-10490 for pig IL-8.

Human IL-8 is produced in various cells, and is reported to be processed differently at the N-terminal (Leonard, E. J. et al., Am. J. Respir. Cell. Mol. Biol. (1990) 2, 479-486). Although human IL-8 having 79, 77, 72, 71, 70 and 69 amino acid residues are known thus far, the number of amino acid residues is not specified in the present invention provided the IL-8 can be used as an antigen for acquisition of anti-IL-8 antibody used in the present invention.

After inserting the gene sequence of IL-8 into a known expression vector system and transforming suitable host cells, the target IL-8 protein is purified by known methods from the host cells or culture supernatant, and this purified IL-8 protein should then be used as sensitizing antigen.

Although there are no particular restrictions on mammals immunized with sensitizing antigen, they are preferably selected in consideration of their compatibility with the parent cells used for cell fusion. In general, animals of the rodent, rabbit and primate orders are used. Examples of animals of the rodent order that are used include mice, rats and hamsters. Examples of animals of the rabbit order that are used include rabbits. Examples of animals of the primate order that are used include monkeys. Examples of monkeys that are used include monkeys of the catarrhine order such as cynomolgus monkeys, rhesus monkeys, hamadryas baboons and chimpanzees.

Immunization of animals with a sensitizing antigen is performed in accordance with known methods. For example, as a general method, immunization is performed by intraperitoneal or subcutaneous injection of sensitizing antigen into the mammal. More specifically, the sensitizing antigen is diluted with PBS (phosphate-buffered saline) or physiological saline and so forth, the resulting suspension is mixed with a suitable amount of ordinary adjuvant as desired, an example of which is Freund's complete adjuvant, and after emulsifying, the sensitizing antigen is administered in several rounds to the animal every 4-21 days. In addition, a suitable carrier can be used during immunization with sensitizing antigen.

After confirming immunization in this manner has resulted in a rise in the desired antibody level in the serum according to routine methods, immunocytes such as lymph node cells or spleen cells are removed from the mammal and used for cell fusion. Spleen cells are particularly preferable examples of immunocytes.

Mammalian myeloma cells used as the corresponding parent cells that are fused with the above-mentioned immunocytes include various known cell lines, examples of which that are used preferably include P3 (P3x63Ag8.653) (Kearney, J. F. et al., J. Immunol. (1979) 123, 1548-1550), P3x63Ag8U.1 (Yelton, D. E. et al., Current Topics in Microbiology and Immunology (1978) 81, 1-7), NS-1 (Kohler, G. and Milstein, C., Eur. J. Immunol. (1976) 6, 511-519), MPC-11 (Margulies, D. H. et al., Cell (1976) 8, 405-415), SP2/0 (Shulman, M. et al., Nature (1978) 276, 269-270), FO (de St. Groth, S. F. and Scheidegger, D., J. Immunol. Methods (1980) 35, 1-21), S194 (Trowbridge, I. S., J. Exp. Med. (1978) 148, 313-323) and R210 (Galfre, G. et al., Nature (1979) 277, 131-133).

Cell fusion of the above-mentioned immunocytes and myeloma cells can basically be carried out in compliance with known methods, an example of which is the method of Milstein, et al. (Galfre, G. and Milstein, C., Methods Enzymol. (1981) 73, 3-46).

More specifically, the above-mentioned cell fusion can be carried out, for example, in an ordinary nutrient culture liquid in the presence of cell fusion promoter. Examples of fusion promoters that are used include polyethylene glycol (PEG) and Sendai virus (HVJ). Moreover, an assistant such as dimethylsulfoxide can be added and used to enhance fusion efficiency as desired.

The ratio of immunocytes and myeloma cells used is preferably, for example, 1-10 times as many immunocytes as myeloma cells. Examples of culture liquids that can be used in the above-mentioned cell fusion include RPMI1640 culture liquid, MEM culture liquid and other culture liquids suitable for propagation of the above-mentioned myeloma cells, as well as ordinary culture liquids used in this type of cell culturing. Moreover, serum supplement such as fetal calf serum (FCS) can also be used in combination with the above.

For cell fusion, prescribed amounts of the above-mentioned immunocytes and myeloma cells are mixed well in the above-mentioned culture liquid followed by addition and mixing of PEG solution, for example PEG solution having a mean molecular weight of about 1000-6000, warmed in advance to about 37° C. and normally having a concentration of 30-60% (w/v), to form the target fused cells (hybridoma). Next, suitable culture liquid is added successively followed by centrifugation and removing the supernatant. By repeating this procedure, cell fusion agents and so forth that are not preferable for hybridoma growth can be removed.

The applicable hybridoma is selected by culturing in ordinary selective culture liquid such as HAT culture liquid (culture liquid containing hypoxanthine, aminopterin and thymidine). Culturing in said HAT culture liquid is continued for an amount of time that is sufficient for destroying cells other than the target hybridoma (non-fused cells), which is usually several days to several weeks. Next, ordinary limiting dilution is performed to screen and clone a hybridoma that produces the target antibody.

In addition, besides obtaining the above-mentioned hybridoma by immunizing animals other than humans with antigen, hybridoma can also be obtained that produces the desired human antibodies having binding activity to IL-8 by sensitizing human lymphocytes to IL-8 in vitro, and fusing the sensitized lymphocytes with human-derived myeloma cells, such as U266, that have the ability to divide indefinitely (see Japanese Examined Patent Publication No. 1-59878). Moreover, human antibody to IL-8 may also be acquired by using a hybridoma in which transgenic animals having a human antibody gene repertoire are immunized with IL-8 serving as antigen to acquire anti-IL-8 antibody-producing cells which are then fused with myeloma cells (see International Patent Application Publication Nos. WO 92/03918, WO 93/12227, WO 94/02602, WO 94/25585, WO 96/33735 and WO 96/34096).

Hybridomas that produce monoclonal antibodies prepared in this manner can be subcultured in ordinary culture liquid, and stored for long periods of time in liquid nitrogen.

In order to acquire monoclonal antibody from these hybridomas, methods are employed such as culturing said hybridoma in accordance with routine methods to obtain monoclonal antibody in the form of the culture supernatant, or transplanting the hybridoma into a mammal that is compatible with it to allow the hybridoma to propagate and obtain monoclonal antibody in the form of the resulting ascites. The former method is suited for obtaining highly pure antibodies, while the latter is suited for large-amount production of antibodies.

In addition to producing antibody using hybridoma, cells may be used in which immunocytes such as sensitized lymphocytes that produce antibody are immortalized by an oncogene.

3. Recombinant Antibody

Monoclonal antibody can also be obtained in the form of recombinant antibody produced using gene recombination technology. For example, recombinant antibody is produced by cloning antibody gene from hybridoma or sensitized lymphocytes or other immunocytes that produce antibody, incorporating in a suitable vector and introducing the vector into a host. This recombinant antibody can be used in the present invention (see, for example, Borrebaeck, C. A. K. and Larrick, J. W., Therapeutic Monoclonal Antibodies, published in the United Kingdom by Macmillan Publishers Ltd., 1990).

More specifically, mRNA that codes for the variable region (V region) of anti-IL-8 antibody is isolated from hybridoma that produces anti-IL-8 antibody. Isolation of mRNA is performed by preparing total RNA according to a known method such as guanidine centrifugation (Chirgwin, J. M. et al., Biochemistry (1979) 18, 5294-5299) or AGPC (Chomczynski, P. and Sacchi, N., Anal. Biochem. (1987) 162, 156-159), and purifying mRNA from the total RNA using, for example, an mRNA Purification Kit (Pharmacia). In addition, mRNA can also be prepared directly by using the QuickPrep mRNA Purification Kit (Pharmacia).

cDNA of the antibody V region is synthesized from the resulting mRNA using reverse transcriptase This can also be performed by using an AMV Reverse Transcriptase First-strand cDNA Synthesis Kit (Biochemical Industries). In addition, the 5′-RACE method (Frohman, M. A. et al., Proc. Natl. Acad. Sci. U.S.A. (1988) 85, 8998-9002; Belyavsky, A. et al., Nucleic Acids Res. (1989) 17, 2919-2932) can be used for performing synthesis and amplification of cDNA using the 5′-Ampli Finder RACE Kit (Clontech) and polymerase chain reaction (PCR).

The target DNA fragment is purified from the resulting PCR product and ligated with vector DNA. Moreover, a recombinant vector is prepared from this, introduced into E. coli and so forth, and colonies are selected to prepare the desired recombinant vector. The base sequence of the target DNA is confirmed by a known method, such as deoxynucleotide chain termination.

If DNA can be obtained that codes for the V region of the target anti-IL-8 antibody, this is ligated with DNA that codes for the desired antibody constant region (C region) and then incorporated in an expression vector. Alternatively, DNA encoding the antibody V region may be incorporated in an expression vector already containing DNA of the antibody C region. Antibody C region derived from the same animal species as the V region, or antibody C region derived from an animal species different from the V region may be used for the antibody C region.

In order to produce the anti-IL-8 antibody used in the present invention, antibody gene is incorporated in an expression vector so that it is expressed under the control of an expression control region such as an enhancer or promoter. Next, host cells are transformed by this expression vector to express antibody.

Expression of antibody gene may be performed by incorporating DNA encoding antibody heavy chain (H chain) or light chain (L chain) into separate expression vectors and then simultaneously transforming host cells, or by incorporating DNA encoding H chain and L chain into a single expression vector and then transforming host cells (see International Patent Application Laid-Open Publication No. WO 94/11523).

4. Altered Antibody

The recombinant antibody used in the present invention can use a altered antibody prepared using genetic engineering techniques for the purpose of lowering heterogeneic antigenicity to humans. Altered antibody has human antibody C region, and chimeric or humanized antibody can be used. These altered antibodies can be produced using known methods.

Chimeric antibodies are obtained by ligating DNA encoding antibody V region other than human antibody obtained in the manner described above with DNA encoding human antibody C region, incorporating this in an expression vector and introducing this into a host to produce chimeric antibody (see European Patent Application Laid-Open Publication No. EP 125023, International Patent Application Laid-Open Publication No. WO 96/02576). Chimeric antibody that is useful in the present invention can be obtained using this known method.

Furthermore, E. coli having a plasmid that contains the L chain or H chain of chimeric WS-4 antibody were internationally deposited based on the Budapest Treaty as FERM BP-4739 and FERM BP-4740, respectively, on Jul. 12, 1994 at the National Institute of Bioscience and Human-Technology the Agency of Industrial Science and Technology (1-1, Higashi 1-chome, Tsukuba-shi, Ibaraki) under the names Escherichia coli DH5α (HEF-chWS4L-gκ) and Escherichia coli JM109 (HEF-chWS4H-gγ1), respectively.

Humanized antibodies are also referred to as reshaped human antibodies. They consist of transplanting the complementarity determining region (CDR) of antibody from a mammal other than humans, such as mouse antibody, to the complementarity determining region of human antibody, and their gene recombination techniques are known (see European Patent Publication No. EP 125023, International Patent Publication No. WO 96/02576).

More specifically, a DNA sequence designed so as to ligate mouse antibody CDR with human antibody framework region (FR) is synthesized by dividing it into a plurality of oligonucleotides having portions that mutually overlap at the ends, and then synthesized to DNA integrated into a single strand by PCR. The resulting DNA is ligated with DNA encoding human antibody C region, and then obtained by incorporating in an expression vector and producing by introducing into a host (see European Patent Publication No. EP 239400, International Patent Publication No. WO 96/02576).

An FR in which CDR forms a good antigen binding site is selected for the FR of the human antibody ligated via the CDR. The amino acids of the FR of the antibody V region may be substituted as necessary so that the complementarity determining region of humanized antibody forms a suitable antigen binding site (Sato, K. et al., Cancer Res. (1993) 53, 851-856).

A specific example of a preferable humanized antibody used in the present invention is humanized WS-4 antibody (see International Patent Publication No. WO 96/02576). In humanized WS-4 antibody, the CDR of mouse-derived WS-4 antibody is ligated with the FR of human antibody REI with respect to the L chain, and with FR1-3 of human antibody VDH26 and FR4 of human antibody 4B4 with respect to the H chain. A portion of the amino acid residues of the FR is substituted so as to have antigen binding activity.

Furthermore, E. coli having a plasmid that contains the L chain or H chain or humanized WS-4 antibody were internationally deposited based on the Budapest Treaty as FERM BP-4738 and FERM BP-4741, respectively, on Jul. 12, 1994 at the National Institute of Bioscience and Human-Technology the Agency of Industrial Science and Technology (1-1, Higashi 1-chome, Tsukuba-shi, Ibaraki) under the names Escherichia coli DH5α (HEF-RVLa-gκ) and Escherichia coli JM109 (HEF-RVHg-gγ1), respectively.

In order to produce the anti-IL-8 antibody used in the present invention, antibody gene is incorporated in an expression vector so that it is expressed under the control of an expression control region such as an enhancer or promoter. Next, host cells are transformed by this expression vector to express antibody.

Expression of antibody gene may be performed by incorporating DNA encoding antibody heavy chain (H chain) or light chain (L chain) into separate expression vectors and then simultaneously transforming host cells, or by incorporating DNA encoding H chain and L chain into a single expression vector and then transforming host cells (see International Patent Publication No. WO 94/11523).

Chimeric antibody is composed of the V region of non-human mammalian antibody and the C region of human-derived antibody. Humanized antibody is composed of the CDR of non-human mammalian antibody, and the FR and C regions of human-derived antibody. Since the amino acid sequences of non-human mammals are reduced to the minimum limit, antigenicity in the human body is decreased thereby making these useful as active ingredients of the therapeutic agent of the present invention.

Examples of human antibody C regions that can be used include Cγ1, Cγ2, Cγ3 and Cγ4. In addition, human antibody C region may be modified to improve antibody or its production stability. For example, in the case IgG4 is selected for the antibody subclass, by converting a portion of the amino acid sequence Cys-Pro-Ser-Cys-Pro of the IgG4 hinge region to the amino acid sequence Cys-Pro-Pro-Cys-Pro of the IgG1 hinge region, the structural instability of IgG4 can be eliminated (Angal, S. et al., Mol. Immunol. (1993) 30, 105-108).

5. Antibody Fragments and Modified Antibodies

The antibody used in the present invention may be an antibody fragment or modified antibody provided is binds to IL-8 and inhibits IL-8 activity. Examples of antibody fragments include Fab, F(ab′)2, Fv or single chain Fv (scFv) in which H chain and L chain Fv are linked with a suitable linker. More specifically, after either treating antibody with an enzyme such as papain or pepsin to form antibody fragments, or after constructing a gene that encodes these antibody fragments and introducing this into an expression vector, they are expressed in suitable host cells (see, for example, Co, M. S. et al., J. Immunol. (1994) 152, 2968-2976; Better, M. and Horwitz, A. H., Methods Enzymol. (1989) 178, 476-496; Pluckthun, A. and Skerra, A., Methods Enzymol. (1989) 178, 497-515; Lamoyi, E., Methods Enzymol. (1986) 121, 652-663; Rousseaux, J. et al., Methods Enzymol. (1986) 121, 663-669; Bird, R. E. and Walker, B. W., Trends Biotechnol. (1991) 9, 132-137).

scFv is obtained by linking antibody H chain V region and L chain V region. In this scFv, H chain V region and L chain V region are linked by means of a linker, and preferably a peptide linker (Huston, J. S. et al., Proc. Natl. Acad. Sci. U.S.A. (1988) 85, 5879-5883). The H chain V region and L chain V region in scFv may be derived from any origin described for the above-mentioned antibodies. An example of a peptide linker that links the V regions is an arbitrary single-strand peptide composed of 12-19 amino acid residues.

DNA encoding scFv is obtained by using DNA encoding H chain or H chain V region of the above-mentioned antibody, and DNA encoding L chain or L chain V region as templates, amplifying the DNA portion encoding the desired amino acid sequence among those sequences by PCR using a primer pair that defines both of its ends, and amplifying by combining DNA encoding a peptide linker portion and a primer pair that defines both of its ends so that each H chain and L chain are linked.

In addition, once DNA encoding scFv is prepared, an expression vector that contains it and a host that is transformed by said expression vector can be obtained in accordance with routine methods. In addition, scFv can be obtained in accordance with routine methods by using that host.

These antibody fragments can be produced in a host by expressing in the same manner as previously described by acquiring its gene. These antibody fragments are also included in the “antibody” referred to in the scope of claim for patent of the present application.

Anti-IL-8 antibody bound to various molecules such as polyethylene glycol (PEG) can be used as modified antibody. These modified antibodies are also included in the “antibody” referred to in the scope of claim for patent of the present application. In order to obtain such modified antibodies, chemical modification is performed on the resulting antibodies. These methods have already been established in this field.

6. Expression and Production of Recombinant Antibody, Altered Antibody or Antibody Fragments

Antibody genes constructed in the manner described above can be expressed and acquired by known methods. In the case of mammalian cells, antibody gene can be expressed in an expression vector containing DNA functionally coupling a commonly used useful promoter/enhancer, the antibody gene to be expressed and a poly A signal downstream on its 3′ side. An example of a promoter/enhancer is human cytomegalovirus immediate early promoter/enhancer.

In addition, virus promoters/enhancers such as retrovirus, polyoma virus, adenovirus and simian virus 40 (SV 40), and mammalian cell-derived promoters/enhancers such as human elongation factor 1α (HEF1α) should be used as other promoters/enhancers that can be used to express the antibody used in the present invention.

For example, in the case of using SV 40 promoter/enhancer, expression of antibody gene can be easily carried out by following the method of Mulligan, R. C. et al. (Nature (1979) 277, 108-114), or in the case of using HEF1α promoter/enhancer, it can be easily carried out by following the method of Mizushima, S. et al. (Nucleic Acids Res. (1990) 18, 5322).

In the case of E. coli, antibody gene can be expressed by functionally coupling a commonly used useful promoter/enhancer, signal sequence for antibody secretion and the antibody gene to be expressed. Examples of promoters include lacZ promoter and araB promoter. In the case of using lacZ promoter, expression should be performed in accordance with the method of Ward, E. S. et al. (Nature (1989) 341, 544-546; Faseb J. (1992) 6, 2422-2427), or in the case of using araB promoter, expression should be performed in accordance with the method of Better, M. et al. (Science (1988) 240, 1041-1043).

The pelB signal sequence (Lei, S. P. et al., J. Bacteriol. (1987) 169, 4379-4383) should be used for the signal sequence for antibody secretion in the case of producing in E. coli periplasm. After separating antibody produced in periplasm, the antibody is used after suitably refolding the antibody structure (see, for example, International Patent Application Laid-Open No. WO 96/30394).

Replication origins derived from SV40, polyoma virus, adenovirus, bovine papilloma virus (BPV) and so forth can be used for the replication origin. Moreover, the expression vector can contain aminoglycoside transferase (APH) gene, thymidine kinase (TK) gene, E. coli xanthine-guanine phosphoribosyl transferase (Ecogpt) gene, dihydrofolate reductase (dhfr) gene and so forth as a selection marker for amplifying the number of gene copies in the host cell system.

An arbitrary production system can be used to produce the antibody used in the present invention, and the production system for antibody production may be an in vitro or in vivo production system.

Examples of in vitro production systems include production systems using eucaryotic cells and production systems using procaryotic cells.

In the case of using eucaryotic cells, examples of production systems include those using animals cells, plant cells and fungal cells. Known examples of animal cells include (1) mammalian cells such as CHO, COS, myeloma, BHK (baby hamster kidney), HeLa and Vero cells, (2) amphibian cells such as Xenopus laevis oocytes, and (3) insect cells such as sf9, sf21 and Tn5 cells. Known examples of plant cells include the genus Nicotiana, and more specifically, Nicotiana tabacum-derived cells, and these cell should be callus cultured. Known examples of fungal cells include (1) yeasts such as the genus Saccharomyces, and more specifically, Saccharomyces cerevisiae, and (2) molds such as the genus Aspergillus, and more specifically, Aspergillus niger.

In the case of using procaryotic cells, examples of production systems include those using bacterial cells. Examples of bacterial cells include Escherichia coli and Bacillus subtilis.

The target antibody gene is introduced into these cells by transformation, and the transformed cells are cultured in vitro to obtain antibody. Culturing is performed in accordance with known methods. For example, DMEM, MEM, RPMI1640 and IMDM can be used as the culture liquid for mammalian cells, and serum supplement such as fetal calf serum (FCS) can be used in combination with these culture liquids. In addition, antibody may also be produced in vivo by transplanting cells containing antibody gene into an animal abdominal cavity and so forth.

Examples of in vivo production systems include production systems using animals and production systems using plants. In the case of using animals, examples of production systems that are used include those using mammals and insects.

Goat, pig, sheep, mouse and cow can be used as mammals (Glaser, V., Spectrum Biotechnology Applications, 1993). In addition, in the case of using mammals, transgenic animals can be used. For example, antibody gene is prepared in the form of a fusion gene by inserting antibody gene into the intermediate portion of a gene encoding a protein characteristically produced in a milk like goat β-casein. A DNA fragment containing fusion gene into which antibody gene has been inserted is introduced into a goat embryo, and this embryo is introduced into a female goat. The desired antibody is then obtained from the milk produced by the transgenic animal born from the goat that received the embryo or its offspring. A suitable hormone may be used in the transgenic goat to increase the amount of milk that contains the desired antibody produced from the transgenic goat (Ebert, K. M. et al., Bio/Technology (1994) 12, 699-702).

In addition, silkworm can be used as an insect. In the case of using silkworm, baculovirus inserted with the target antibody gene is infected into silkworm, and the desired antibody is obtained from the body fluid of this silkworm (Maeda, S. et al., Nature (1985) 315, 592-594).

Moreover, in the case of using plants, tobacco plant, for example, can be used. In the case of using tobacco plant, the target antibody gene is inserted into a vector for plant expression such as pMON 530, and this vector is introduced into a bacteria like Agrobacterium tumefaciens. This bacteria is infected into a tobacco plant, for example, Nicotiana tabacum, to obtain the desired antibody from the leaves of the mature tobacco plant (Ma, J. K. et al., Eur. J. Immunol. (1994) 24, 131-138).

Antibody gene like that described above is introduced into these animals or plants, and antibody is produced inside the animal or plant body and recovered.

In the case of producing antibody with an in vitro or in vivo production system as described above, DNA encoding antibody heavy chain (H chain) or light chain (L chain) may be incorporated into separate expression vectors followed by simultaneous transformation of a host. Alternatively, DNA encoding H chain and L chain may be incorporated into a single expression vector followed by transformation of a host (see International Patent Application Laid-Open Publication No. WO 94/11523).

7. Antibody Separation and Purification

Antibody expressed and produced in the manner described above can be separated from the host inside and outside cells and purified to uniformity. Separation and purification methods used with ordinary proteins should be used for the separation and purification of antibody used in the present invention, and there are no limitations on these methods whatsoever. For example, antibody can be separated and purified by suitably selecting and combining a chromatography column used in affinity chromatography and so forth, filter, ultrafiltration, salting out, dialysis and so forth (Antibodies: A Laboratory Manual. Ed Harlow and David Lane, Cold Spring Harbor Laboratory Press, 1988). Examples of columns used in affinity chromatography include a protein A column and protein G column. Examples of columns that use a protein A column include Hyper D, POROS and Sepharose F.F. (Pharmacia). Examples of chromatography methods other than affinity chromatography include ion exchange chromatography, hydrophobic chromatography, gel filtration, reverse phase chromatography and adsorption chromatography (Strategies for Protein Purification and Characterization: A Laboratory Course Manual. Ed. Daniel R. Marshak, et al., Cold Spring Harbor Laboratory Press, 1996). Moreover, these chromatographies can be performed using liquid phase chromatography such as HPLC and FPLC.

8. Measurement of Antibody Concentration

Measurement of the concentration of the antibody obtained as described above can be performed by measurement of absorbance or enzyme-linked immunosorbent assay (ELISA) and so forth. Namely, in the case of measuring concentration by measuring absorbance, after suitably diluting the resulting antibody with PBS, the absorbance at 280 nm is measured, and although the absorption coefficient differs according to the species and subclass, absorbance is calculated using an OD of 1.4 for 1 mg/ml in the case of human antibody. In addition, in the case of measuring concentration by ELISA, measurement can be performed in the manner described below. Namely, 100 μl of goat anti-human IgG antibody diluted to 1 μg/ml with 0.1 M bicarbonate buffer (pH 9.6) are added to a 96-well plate (Nunc), and the plate is incubated overnight at 4° C. to convert the antibody to a solid phase. After blocking, 100 μl of suitably diluted antibody used in the present invention or sample containing antibody, or a known concentration of human IgG used as a concentration standard are added followed by incubation for 1 hour at room temperature. After washing, 100 μl of 5000-fold diluted alkaline phosphatase-labeled anti-human IgG antibody are added followed by incubating for 1 hour at room temperature. After washing, substrate solution is added and following incubation, absorbance is measured at 405 nm using a Microplate Reader Model 3550 (Bio-Rad), and the concentration of target antibody is calculated from the absorbance of the concentration standard human IgG.

9. Confirmation of Antibody Activity

Known means can be used to measure the antigen binding activity (Antibodies: A Laboratory Manual. Ed Harlow and David Lane, Cold Spring Harbor Laboratory Press, 1988) and ligand receptor binding inhibitory activity (Harada, A. et al., Int. Immunol. (1993) 5, 681-690) of the antibody used in the present invention.

ELISA, EIA (enzyme immunoassay), RIA (radioimmunoassay) or fluorescent antibody methods can be used as methods for measuring the antigen binding activity of the anti-IL-8 antibody used in the present invention.

For example, in the case of using ELISA, IL-8 is added to a 96-well plate in which polyclonal antibody to IL-8 is in the solid phase, and a sample containing the target anti-IL-8 antibody, such as a culture supernatant of anti-IL-8 antibody-producing cells or purified antibody, is added. Secondary antibody, which is labeled with an enzyme such as alkaline phosphatase and recognizes the target anti-IL-8 antibody, is added. After incubating the plate and washing, an enzyme substrate such as p-nitrophenyl phosphate is added followed by measurement of absorbance to assess antigen binding activity.

Ordinary Cell ELISA or ligand receptor binding assay can be used as a method for measuring ligand receptor binding inhibitory activity of the -anti-IL-8 antibody used in the present invention.

For example, in the case of using Cell ELISA, blood cells or cancer cells that express IL-8 receptor, such as neutrophils, are cultured in a 96-well plate and adhered to the plate, followed by fixation with paraformaldehyde, etc. Alternatively, a solid-phase 96-well plate is prepared by preparing a membrane fraction of cells that express IL-8 receptor. A sample containing the target anti-IL-8 antibody, such as the culture supernatant of anti-IL-8 antibody-producing cells or purified antibody, and IL-8 labeled with a radioisotope such as ¹²⁵I are added to the above-mentioned plate. After incubating the plate and washing, radioactivity is measured to measure the amount of IL-8 bound to IL-8 receptors and assess the ligand receptor binding inhibitory activity of anti-IL-8 antibody. For example, for a binding inhibition assay of IL-8 to cellular IL-8 receptors, after separating blood cells or cancer cells, such as neutrophils, that express IL-8 receptor by means such as centrifugal separation, the cells are prepared in the form of a cell suspension. A solution of IL-8 labeled with a radioisotope such as ¹²⁵I or a mixed solution of non-labeled IL-8 and labeled IL-8, and a solution containing concentration-adjusted anti-IL-8 antibody are added to the cell suspension. After a predetermined amount of time, the cells are separated, and the radioactivity of labeled IL-8 bound on the cells should then be measured.

In addition, known routine methods, such as the method of Grob, P. M. et al. (J. Biol. Chem. (1990) 265, 8311-8316), can be used as a method for measuring the ability of anti-IL-8 antibody used in the present invention to inhibit neutrophil chemotaxis.

More specifically, after diluting anti-IL-8 antibody with a culture liquid such as RPMI1640, DMEM, MEM or IMDM, IL-8 is added and this is then poured into the bottom layer of the chamber separated with a filter using a commercially available chemotaxis chamber. Next, a prepared cell suspension, such as a neutrophil suspension, is added to the top layer of the chamber after which the chamber is allowed to stand for a predetermined amount of time. Since migrating cells adhere to the bottom surface of the filter installed in the chamber, the number of those cells should then be measured with a method using a staining liquid or fluorescent antibody and so forth. In addition, this can also be performed by making a judgment based on a visual assessment under a microscope or by automated measurement using a measuring instrument.

10. Administration Methods and Preparations

A therapeutic agent containing as its active ingredient the anti-IL-8 antibody of the present invention can be parenterally administered either generally or locally by, for example, intravenous injection such as intravenous infusion, intramuscular injection, intraperitoneal injection or subcutaneous injection. In addition, a suitable administration method can be selected according to the patient's age and symptoms.

A therapeutic agent containing as its active ingredient the anti-IL-8 antibody of the present invention is administered to patients already suffering from illness at a dose level that is sufficient for either remedying the symptoms of the illness or at least partially inhibiting those symptoms. For example, the effective dose level is selected over a range of 0.01 mg to 1000 mg per kg of body weight per administration. Alternatively, a dose level of 5 to 2000 mg/body per patient can also be selected. However, the therapeutic agent containing as its active ingredient the anti-IL-8 antibody of the present invention is not limited to these dose levels.

In addition, for the time of administration, the therapeutic agent of the present invention may be administered after the occurrence of sepsis or septic shock, or when the occurrence of sepsis or septic shock is predicted.

In addition, the period of administration can be suitably selected according to the patient's age and symptoms.

A therapeutic agent containing as its active ingredient the anti-IL-8 antibody of the present invention can be prepared in accordance with routine methods (Remington's Pharmaceutical Science, Latest Edition, Mark Publishing Company, Easton, U.S.A.), and may also contain a pharmacologically allowed carrier or additive.

Examples of such carriers or pharmaceutical additives include water, pharmacologically allowed organic solvents, collagen, polyvinyl alcohol, polyvinyl pyrrolidone, carboxyvinyl polymer, sodium carboxymethyl cellulose, sodium polyacrylate, sodium arginate, water-soluble dextran, sodium carboxymethyl starch, pectin, methyl cellulose, ethyl cellulose, xanthan gum, gum arabic, casein, agar, polyethylene glycol, diglycerin, glycerin, propylene glycol, Vaseline, paraffin, stearyl alcohol, stearic acid, human serum albumin (HSA), mannitol, sorbitol, lactose and pharmacologically allowed surface activators.

Although actual additives are suitably selected or combined from among those listed above according to the drug form of the therapeutic agent of the present invention, they are naturally not limited to these.

For example, in the case of using the therapeutic agent of the present invention as an injection preparation, purified anti-IL-8 antibody is dissolved in a solvent such as physiological saline, buffer or glucose solution, and can be used following the addition of an adsorption preventer such as Tween 80, Tween 20, gelatin or human serum albumin. Alternatively, the pharmaceutical preparation may be freeze-dried in order to be reconstituted prior to use. Examples of vehicles for freeze-drying include sugar alcohols and sugars such as mannitol and glucose.

Sepsis is a disease having clinical findings that are all two or more among the four diagnostic parameters of the above-mentioned SIRS that is caused by infection. The pathogen that causes the infection may or may not be confirmed. Trauma, burns and severe pancreatitis are distinguished from sepsis in that the direct cause is not infection. In addition, septic shock is a disease accompanied by perfusion abnormalities such as low blood pressure even though an adequate amount of circulating body fluids is maintained. As sepsis progresses, there is onset of septic shock within several hours, presenting with decreased systemic peripheral vascular resistance, decreased myocardial contractile force, peripheral circulatory insufficiency, decreased blood pressure and so forth. As indicated in the examples below, a therapeutic agent containing as its active ingredient the anti-IL-8 antibody of the present invention inhibits decreased arterial pressure, increased respiration rate and changes in body temperature with administration of endotoxin to rabbits known as experimental systems for the above-mentioned diseases, while also improving the survival rate of rabbits administered with endotoxin.

Thus, a therapeutic agent containing for its active ingredient the anti-IL-8 antibody of the present invention is useful as a therapeutic agent for sepsis and septic shock. In addition, a therapeutic agent containing for its active ingredient the anti-IL-8 antibody of the present invention is useful in improving decreased arterial pressure during septic shock as well as relieving increased respiration rate during septic shock.

### EXAMPLES

Although the following provides a detailed explanation of the present invention through its examples and reference examples, the present invention is not limited by these.

### Reference Example 1

### Preparation of Hybridoma Producing Monoclonal Antibody to Human IL-8

BALB/c mice were immunized with human IL-8 in accordance with routine methods, and spleen cells were sampled from immune mice. These spleen cells were fused with mouse myeloma cells P3X63Ag8.653 in accordance with routine methods using polyethylene glycol to prepare hybridoma that produces mouse monoclonal antibody to human IL-8 antibody. As a result of screening using binding activity to human IL-8 as an indicator, hybridoma cell line WS-4 was obtained. In addition, antibody produced by hybridoma WS-4 had neutralizing activity that inhibited binding of neutrophils by human IL-8 (Ko, Y. et al., J. Immunol. Methods (1992) 149, 227-235).

The isotypes of the H and L chains of antibody produced by hybridoma WS-4 were investigated using a mouse monoclonal antibody isotyping kit. As a result, antibody produced by hybridoma WS-4 was clearly shown to have mouse κ-type L chain and mouse γ-type H chain.

Furthermore, hybridoma cell line WS-4 was internationally deposited based on the Budapest Treaty as FERM BP-5507 on Apr. 17, 1996 at the National Institute of Bioscience and Human-Technology the Agency of Industrial Science and Technology (1-1-3 Tsukuba, Ibaraki prefecture) under the name Mouse hybridoma WS-4.

### Reference Example 2

### Preparation of Humanized Antibody to Human IL-8

Humanized WS-4 antibody was prepared according to the method described in International Patent Application Laid-Open No. 96/02576. Total RNA was prepared in accordance with routine methods from hybridoma WS-4 prepared in Reference Example 1, and single-strand cDNA was prepared from this total RNA. DNA encoding the V regions of the H chain and L chain of mouse WS-4 antibody was amplified by PCR. The primer used for PCR was the primer described in Jones, S. T. and Bendig, M. M., Bio/Technology (1991) 9, 88-89. The amplified DNA fragment was purified by PCR followed by isolation of a DNA fragment containing a gene encoding the L chain V region of mouse WS-4 antibody, and a DNA fragment containing a gene encoding the H chain V region of mouse WS-4 antibody. These DNA fragments were respectively linked to a plasmid pUC-type cloning vector and introduced into E. coli competent cells to obtain an E. coli transformant.

This transformant was cultured in accordance with routine methods, and a plasmid containing the above-mentioned DNA fragment was purified from the resulting microorganisms. The base sequence of DNA encoding the V region in the plasmid was determined in accordance with routine methods, and each CDR of the V region was identified from its amino acid sequence.

In order to prepare a vector expressing chimeric WS-4 antibody, cDNA encoding the V regions of the L chain and H chain of mouse WS-4 antibody were separately inserted into an HEF vector to which DNA encoding human C region had been ligated in advance.

In order to prepare humanized WS-4 antibody, CDR of the V region of mouse WS-4 antibody was transplanted into human antibody using genetic engineering techniques according to the CDR-grafting method. Substitution of the DNA sequence was performed in order to substitute a portion of the amino acids of the FR of the V region of the antibody to which CDR was transplanted to form a suitable antigen binding site.

DNA respectively encoding the V regions of the L chain and H chain of humanized WS-4 antibody prepared in this manner was separately inserted into an HEF vector to express as antibody in mammalian cells and prepare vectors that express the L chain or H chain of humanized WS-4 antibody.

By simultaneously inserting these two expression vectors into COS cells, a cell line was established that produces humanized WS-4 antibody. The binding ability to IL-8 and the IL-8 neutralizing ability of the humanized WS-4 antibody obtained by culturing this cell line were respectively investigated by ELISA and an IL-8/neutrophil binding inhibition test. As a result, humanized WS-4 antibody bound to human IL-8 to the same degree as mouse WS-4 antibody, and was determined to inhibit binding of IL-8 to neutrophils.

Furthermore, E. coli having a plasmid containing the L chain and H chain of humanized WS-4 antibody are internationally deposited based on the Budapest Treaty as FERM BP-4738 and FERM BP-4741, respectively, on Jul. 12, 1994 at the National Institute of Bioscience and Human-Technology the Agency of Industrial Science and Technology (1-1, Higashi 1-chome, Tsukuba-shi, Ibaraki) under the names Escherichia coli DH5α (HEF-RVLa-gκ) and Escherichia coli JM109 (HEF-RVHg-gγl), respectively.

### Example 1

New Zealand white rabbits (females, 5 per group, body weights: 2.8 to 3.2 kg) were pre-anesthetized by intramuscular administration of 0.5 mg/kg body weight of diazepam and 35 mg/kg body weight of pentobarbital. After allowing to remain undisturbed for 30 minutes, a 24G Telmo catheter was inserted into an auricular vein and the animals were anesthetized by administration of 5 mg/kg body weight of Ketamine through this venous catheter. Next, a 22G Telmo catheter was inserted into an auricular artery.

The following procedure was then performed until termination of anesthesia. (i) Ketamine was additionally injected at the rate of 20 mg/kg body weight per hour through the venous catheter. (ii) Physiological saline was injected at the rate of 5 ml/kg body weight per hour through the venous catheter. (iii) Arterial pressure was measured continuously using the arterial catheter. (iv) Blood samples were periodically collected from the arterial catheter. (v) 2.5 IU/ml of heparin were injected at the rate of 1 ml/kg body weight per hour through the arterial catheter to prevent the catheter from clogging. (vi) Respiration rate and rectal temperature were measured periodically.

The animals were allowed to remain undisturbed for 45 minutes after completion of catheter insertion followed by measurement of baseline arterial pressure, respiration rate and rectal temperature. Immediately after, mouse WS-4 antibody to human IL-8 at the rate of 3 mg/kg body weight, mouse P3.6.2.8.1 antibody as control antibody at the rate of 3 mg/kg body weight, or physiological saline at the rate of 1.8 ml/kg body weight was administered through the venous catheter. Starting 5 minutes later and lasting for 20 minutes, 0.5 mg/kg body weight of lipopolysaccharide (LPS, Escherichia coli 0127:B8, Sigma) or 2 ml/kg body weight of physiological saline was administered through the venous catheter.

Furthermore, the experimental groups were divided into an anti-IL-8 antibody dose group, control antibody dose group, LPS group and normal group. Animals of the anti-IL-8 antibody dose group were administered mouse WS-4 antibody at 0 minutes, and LPS from 5 to 25 minutes. Animals of the control antibody dose group were administered mouse P3.6.2.8.1 antibody at 0 minutes and LPS from 5 to 25 minutes. Animals of the LPS group were administered physiological saline only at 0 minutes and LPS from 5 to 25 minutes. Animals of the normal group were administered physiological saline only both at 0 minutes and from 5 to 25 minutes.

Anesthesia and measurement of each parameter was completed 4 hours after LPS administration, and each animal was returned to the same cage as prior to the experiment. The animals were then evaluated for survival rate until 7 days later.

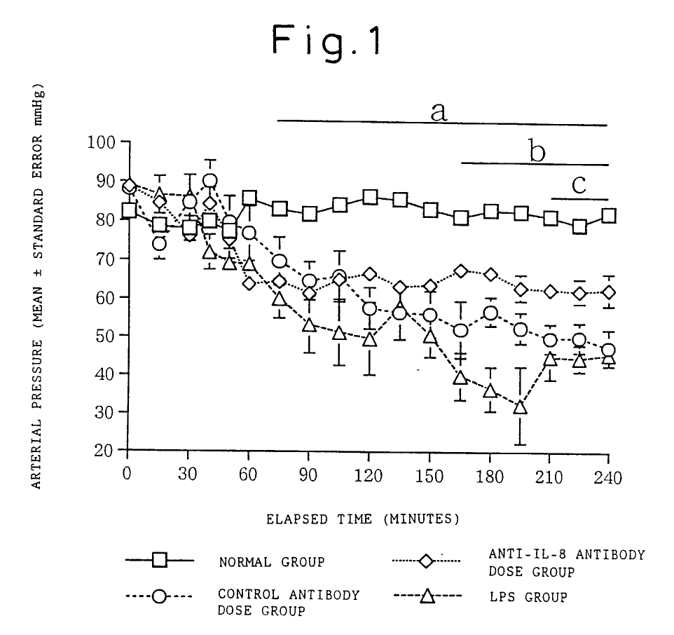

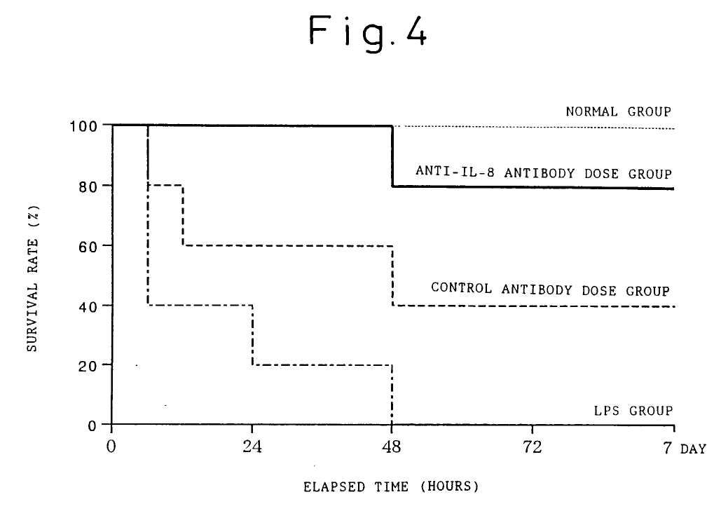

Time-based changes in arterial pressure, respiration rate and rectal temperature are respectively shown in FIGS. 1, 2 and 3. In addition, time-based changes in survival rate are shown in FIG. 4.

(1) Arterial Pressure

In each of the groups administered LPS (anti-IL-8 antibody dose group, control antibody dose group and LPS group), arterial pressure decreased significantly (p<0.05) in comparison with the normal group, and symptoms of shock caused by decreased arterial pressure were indicated due to administration of LPS. However, in the anti-IL-8 antibody dose group, the decrease in arterial pressure was significantly (p<0.05) relieved in comparison with the control antibody group and LPS group.- In addition, there was no significant difference in arterial pressure observed between the control antibody dose group and LPS group (see FIG. 1). Based on these findings, anti-IL-8 antibody was shown to relieve decreased blood pressure, which is one of the symptoms of sepsis and septic shock.

(2) Respiration Rate

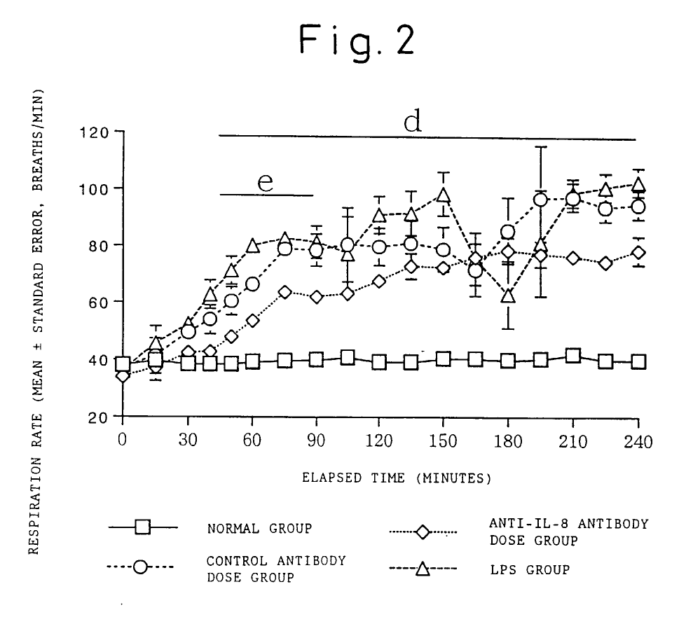

In each of the groups administered LPS (anti-IL-8 antibody dose group, control antibody dose group and LPS group), respiration rate increased significantly (p<0.05) in comparison with the normal group (with the exception of the control antibody dose group at 165 minutes), and increased respiration rate, which is one of the diagnostic parameters of SIRS, was indicated. However, in the anti-IL-8 antibody dose group, increases in respiration rate tended to be relieved in comparison with the control antibody dose group and LPS group, and particularly from 45 to 90 minutes, significant (p<0.05) relief of increased respiration rate was observed in comparison with the LPS group. In addition, there was no significant difference observed in respiration rate between the control antibody dose group and LPS group (see FIG. 2). Based on these findings, anti-IL-8 antibody was shown to relieve increased respiration rate, which is one of the symptoms of sepsis and septic shock.

(3) Rectal Temperature

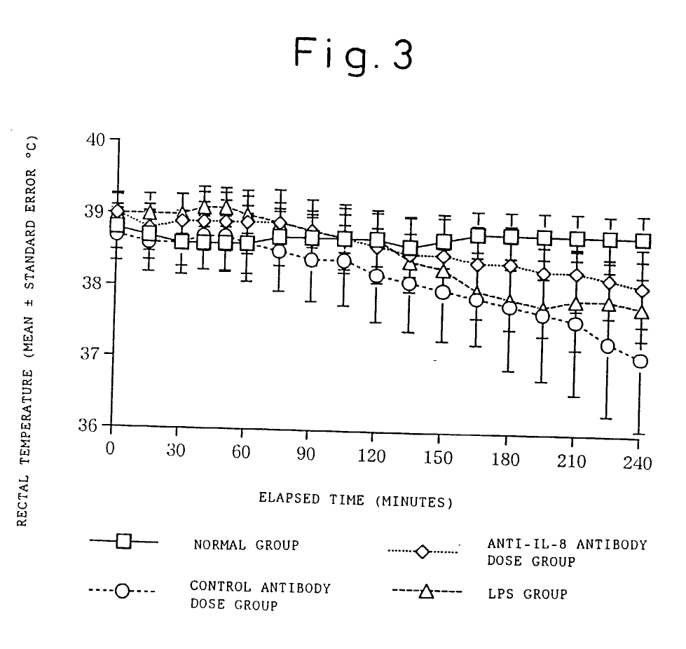

In each group administered LPS (anti-IL-8 antibody dose group, control antibody dose group and LPS group), although there were no statistically significant differences observed, rectal temperature tended to decrease in comparison with the normal group. At that time, in the anti-IL-8 antibody dose group, decreased body temperature tended to be relieved in comparison with the control antibody dose group and LPS group (see FIG. 3). Based on these findings, anti-IL-8 antibody was suggested to relieve decreased body temperature, which is one of the diagnostic parameters of sepsis and septic shock.

(4) Survival Rate

All five animals in the LPS group died by 48 hours after administration and 2 of 5 animals survived in the control antibody dose group at 7 days after administration. In contrast, 4 of 5 animals survived in the anti-IL-8 antibody dose group at 7 days after administration (see FIG. 4). Based on these findings, anti-IL-8 antibody was shown to save the animals from death caused by administration of LPS. Furthermore, all of the animals in the normal group not administered LPS survived.

On the basis of the above, as has been indicated in (1) through (4), anti-IL-8 antibody relieved decreased blood pressure, increased respiration rate and decreased body temperature, which are symptoms of sepsis, including septic shock. Moreover, it also saved animals from death caused by administration of endotoxin.

Industrial Applicability

Anti-IL-8 antibody relieved decreased blood pressure, increased respiration rate and decreased body temperature caused by bacterial toxin, and saved animals from death due to bacterial toxin. These facts indicate that anti-IL-8 antibody is useful as an agent for treatment of sepsis and septic shock, an agent for improvement of decreased arterial pressure, and an agent for relief of increased respiration rate.

Names and Addresses of Depository Institutions at which Microorganisms are Deposited Based on Provision 13 Bis of the Patent Cooperation Treaty

National Institute of Bioscience and Human-Technology Agency of Industrial Science and Technology 1-1, Higashi 1-chome, Tsukuba-shi, Ibaraki

| Deposition Number | Deposition Date |
|---|---|
| FERM BP-4738 | Jul. 12, 1994 |
| FERM BP-4739 | Jul. 12, 1994 |
| FERM BP-4740 | Jul. 12, 1994 |
| FERM BP-4741 | Jul. 12, 1994 |
| FERM BP-5507 | Apr. 17, 1996 |

1. A therapeutic agent for sepsis comprising as its active ingredient an anti-IL-8 antibody.

2. A therapeutic agent as set forth in claim 1 wherein the sepsis is septic shock.

3. A therapeutic agent as set forth in claim 1 or 2 wherein the anti-IL-8 antibody is a monoclonal antibody.

4. A therapeutic agent as set forth in any one of claims 1 through 3 wherein the anti-IL-8 antibody is an antibody to mammalian IL-8.

5. A therapeutic agent as set forth in any one of claims 1 through 4 wherein the anti-IL-8 antibody is antibody to human IL-8.

6. A therapeutic agent as set forth in any one of claims 1 through 5 wherein the anti-IL-8 antibody is WS-4 antibody.

7. A therapeutic agent as set forth in any one of claims 1 through 6 wherein the anti-IL-8 antibody comprises human antibody constant region.

8. A therapeutic agent as set forth in any one of claims 1 through 7 wherein the anti-IL-8 antibody is a humanized or chimeric antibody.

9. A therapeutic agent as set forth in any one of claims 1 through 8 wherein the anti-IL-8 antibody is humanized WS-4 antibody.

10. An agent for improving decreased arterial pressure by septic shock comprising as its active ingredient an anti-IL-8 antibody.

11. An agent for relieving an increased respiration rate of septic shock comprising as its active ingredient an anti-IL-8 antibody.

12. A use of anti-IL-8 antibody for producing a therapeutic agent for sepsis.

13. A use as set forth in claim 12 wherein the sepsis is septic shock.

14. A use as set forth in claim 12 or 13 wherein the anti-IL-8 antibody is a monoclonal antibody.

15. A use as set forth in any one of claims 12 through 14 wherein the anti-IL-8 antibody is an antibody to mammalian IL-8.

16. A use as set forth in any one of claims 12 through 15 wherein the anti-IL-8 antibody is an antibody to human IL-8.

17. A use as set forth in any one of claims 12 through 16 wherein the anti-IL-8 antibody is WS-4 antibody.

18. A use as set forth in any one of claims 12 through 17 wherein the anti-IL-8 antibody comprises a human antibody constant region.

19. A use as set forth in any one of claims 12 through 18 wherein the anti-IL-8 antibody is a humanized or chimeric antibody.

20. A use as set forth in any one of claims 12 through 19 wherein the anti-IL-8 antibody is humanized WS-4 antibody.

21. A use of anti-IL-8 antibody as an agent for improving decreased arterial pressure by septic shock.

22. A use of anti-IL-8 antibody for producing an agent for relieving increased respiration rate of septic shock.

23. A treatment method for sepsis comprising the administration of anti-IL-8 antibody to subjects requiring treatment.

24. A treatment method as set forth in claim 23 wherein the sepsis is septic shock.

25. A treatment method as set forth in claim 23 or 24 wherein the anti-IL-8 antibody is a monoclonal antibody.

26. A treatment method as set forth in any one of claims 23 through 25 wherein the anti-IL-8 antibody is an antibody to mammalian IL-8.

27. A treatment method as set forth in any one of claims 23 through 26 wherein the anti-IL-8 antibody is an antibody to human IL-8.

28. A treatment method as set forth in any one of claims 23 through 27 wherein the anti-IL-8 antibody is WS-4 antibody.

29. A treatment method as set forth in any one of claims 23 through 28 wherein the anti-IL-8 antibody comprises a human antibody constant region.

30. A treatment method as set forth in any one of claims 23 through 29 wherein the anti-IL-8 antibody is a humanized antibody or chimeric antibody.

31. A treatment method as set forth in any one of claims 23 through 30 wherein the anti-IL-8 antibody is humanized WS-4 antibody.

32. A method for improving a decreased arterial pressure by septic shock comprising administration of an anti-IL-8 antibody to subjects requiring treatment.

33. A method for relieving an increased respiration rate by septic shock comprising administration of anti-IL-8 antibody to subjects requiring treatment.
